(19) United States
(12) Patent Application Publication
(10) Pub. No.: US 2009/0116701 A1
(43) Pub. Date: May 7, 2009

(54) SECURE PUPIL RESPONSE TESTING

(75) Inventors: Barry Sim Hochfield, Glasgow (GB); Michelle Govan, Glasgow (GB)

Correspondence Address: FOLEY AND LARDNER LLP;SUITE 500, 3000 K STREET NW, WASHINGTON, DC, 20007, US

(73) Assignee: Ecebs Group Limited

(21) Appl. No.: 11/917,793

(22) PCT Filed: Jun. 22, 2006
(86) PCT No.: PCT/GB2006/002292

§ 371 Date: Jun. 3, 2008

(30) Foreign Application Priority Data
Jun. 22, 2005 (GB) 0512729.5

(51) Int. Cl. A61B 3/11; G06K 9/22

(52) U.S. Cl. 382/117; 351/209

(57) ABSTRACT

In a system for carrying out pupil response testing, an individual who is to be tested is provided with a portable programmable device, for example, a smartcard carrying data representing personal information, for example, the age and gender of the holder. An interface device comprises means for reading the said data from the programmable device, such as a smartcard reader, and test means including a camera for carrying out pupil response testing. Control means process the results from the pupil response test in dependence on the data read from the programmable device by the interface device. Preferably, the smartcard or other programmable device also carries biometric data identifying the individual to whom the programmable device has been issued and the interface device is operable to read the biometric data from the smartcard and verifying that the holder is the individual to whom the smartcard has been issued. The camera which forms part of the pupil response testing means is used to perform the biometric scan, for example, an iris scan, of the holder for verification purposes.

For safety critical tasks, some employees are subject to optical pupil response tests. These tests measure the time to respond to changes in lighting, and can screen out persons whose physical reaction times are not as required to, say, operate safety critical equipment i.e. as to if they are fit for work.

Normal human pupil response times have been studied and are well understood. They vary primarily with a person's age and gender. While it is possible to have an average template against which times are measured, the percentage of false accepts and rejects can be significantly reduced if the template is normalised against the age and gender of the person under test.

To ensure the correct normal profile for the individual being tested is selected, it would be necessary to ascertain the correct age and gender for each test. Manual entry of this data may not be trustworthy if the persons involved have reason to try to ‘fool the test’.

In accordance with the invention there is provided a system for carrying out pupil response testing, the system comprising a portable programmable device carrying data representing personal information relating to an individual who holds the programmable device; an interface device comprising means for reading the said data from the programmable device, test means including a camera for carrying out pupil response testing, and control means operable to process the results from the test means in dependence on the data read from the programmable device by the interface device.

Preferably, the programmable device further comprises biometric data identifying the individual to whom the programmable device has been issued, the interface device is operable to read the said biometric data and the system includes means for carrying out a scan of the holder of the programmable device to verify that the holder is the individual to whom the programmable device has been issued.

In a preferred embodiment, the camera which forms part of the test means is used to perform the scan of the holder of the programmable device to allow the system to verify that the holder is the individual to whom the programmable device has been issued.

In a scheme in accordance with the invention, which is described below by way of example, the person under test must, when the pupil response test is taking place, present a trusted token to an IT system to which the pupil test rig is attached and controlled. The token may, for example, be in the form of a portable programmable device such as a smartcard.

Possession of the token authenticates the individual to be tested (in the same way that possession of a banker's card authenticates its user). The token used in this scheme, however, also contains data indexing the correct age and gender of the individual holder against the normal pupil response template. This prevents the individual giving false information regarding age to the tester.

It will be appreciated that such a system may still be open to abuse in the case where the token is given to a stand-in of the same gender and similar age to undergo testing on behalf of the true holder of the token. Accordingly, it is proposed that, in addition to the basic data relating to age and gender, the smartcard token also carried biometric data uniquely identifying the true holder of the token, conveniently in this case, data from an iris scan.

The testing apparatus of the invention preferably performs biometric authentication of the person under test by carrying out an iris scan and checking the results of that scan against the biometric data held on the smartcard token. The required pupil response test can then be carried out with a much higher level of certainty that the individual being tested is the correct individual than has hitherto been the case.

Both iris scanning and the pupil response test are carried out using apparatus which utilises a camera to provide information to the tester or to an automated test centre.

In a preferred embodiment of the scheme of the invention, the same camera is used, with appropriate control apparatus, to carry out both the iris scan and the pupil response test.

It would also be advantageous to be carry out pupil response testing at remote locations over a computer network or the internet and, in order to achieve this, the camera used is preferably a ‘webcam’ class camera which can be installed at any testing station; allowing a testing station to be set up using a camera, a smartcard reader and a PC with appropriate software.

For the pupil response test, the camera used needs a frame rate of 60-100 frames per second and a pixel area of around 1280×960, pixel depth and appropriate camera control. One model of camera currently available which has the necessary characteristics is the ‘Philips SPC 900NC Pixel Plus 1.3 MP’ webcam, which has video resolution VGA (640×480), a snapshot resolution 1.3 MP (1240×960), a maximum frame rate of 90 fps at CIF and 75 fps at VGA, as well as colour depth 24 bits. The camera is connected to the PC via a conventional USB port. While these features alone provide the minimum required for both iris scanning and pupil response testing, the camera in question also has a ‘Digital Natural Motion’ function to give smooth, judder free iomages and ‘Automatic Face Tracking’ to allow for more freedom of movement on the part of the individual being tested.

The same camera can, thus, be used for both the biometric iris scan and the pupil response scan, speeding up the process and permitting testing to be aarried out at remote locations. It will be appreciated that if other optical biometric scanning techniques were to be used, in place of the iris scanning referred to above, the camera could be used to carry out those scans instead of the iris scanning described

1. A system for carrying out pupil response testing, the system comprising a portable programmable device carrying data representing personal information relating to an individual who holds the programmable device; an interface device comprising means for reading the said data from the programmable device, test means including a camera for carrying out pupil response testing, and control means operable to process the results from the test means in dependence on the data read from the programmable device by the interface device.

2. A system according to claim 1 wherein the programmable device further comprises biometric data identifying the individual to whom the programmable device has been issued, the interface device is operable to read the said biometric data and wherein the system includes means for carrying out a scan of the holder of the programmable device to verify that the holder is the individual to whom the programmable device has been issued.

3. A system according to claim 2 wherein the camera which forms part of the test means is used to perform the scan of the holder of the programmable device to allow the system to verify that the holder is the individual to whom the programmable device has been issued.

4. A system according to claim 2 wherein the biometric data represents an irisscan of the individual.

5. A system according to claim 1 wherein the portable programmable device is a smartcard.

6. An interface device for use in the system of claim 1, the interface device comprising an interface device comprising means for reading data from a portable programmable device, test means including a camera for carrying out pupil response testing, and control means operable to process the results from the test means in dependence on the data read from the programmable device by the interface device.

7. An interface device according to claim 6, the interface device being operable to read biometric data from a portable programmable device and including means for carrying out a scan of the holder of the programmable device to verify by comparing data from the said scan with the said biometric data that the holder is the individual to whom the programmable device has been issued.

8. An interface device according to claim 7 wherein the camera which forms part of the test means is used to perform the scan of the holder of the programmable device to allow the system to verify that the holder is the individual to whom the programmable device has been issued.

9. A interface device according to claim 7 wherein the biometric data represents an irisscan of the individual.

10. An interface device according to claim 6 wherein the means for reading data from a portable programmable device is a smartcard reader.
